(19) United States
(12) Patent Application Publication
(10) Pub. No.: US 2022/0274912 A1
(43) Pub. Date: Sep. 1, 2022

(54) METAL OXIDE FILM-FORMING COMPOSITION, METHOD FOR PRODUCING METAL OXIDE FILM USING THE SAME, TERTIARY ALKYLOXYCARBONYL GROUP-MODIFIED BISNAPHTHOL FLUORENE COMPOUND, AND METHOD FOR PRODUCING THE SAME

(71) Applicant: TOKYO OHKA KOGYO CO., LTD., Kawasaki-shi (JP)

(72) Inventors: Kunihiro NODA, Kawasaki-shi (JP); Takanori KOBAYASHI, Kawasaki-shi (JP); Hiroki KATO, Kawasaki-shi (JP); Dai SHIOTA, Kawasaki-shi (JP)

(21) Appl. No.: 17/652,005

(22) Filed: Feb. 22, 2022

(30) Foreign Application Priority Data
Feb. 26, 2021 (JP) 2021-030463

(51) Int. Cl. C07C 69/96; C01G 23/04; C07C 43/303; C07C 39/17

(52) U.S. Cl. CPC C07C 69/96; C01G 23/04; C07C 43/303; C07C 39/17; C07C 2603/18

(57) ABSTRACT

A metal oxide film-forming containing a tertiary alkyloxycarbonyl group-modified bisnaphthol fluorene compound represented by formula (1), a metal compound represented by formula L(R⁶)ₙ₁(O)ₙ₂, and a solvent. In the formulas, ring Z¹ represents a naphthalene ring; R^{1a} and R^{1b} represent a halogen atom; R^{2a} and R^{2b} represent an alkyl group; R^{3a} to R^{5b} represent an alkyl group having 1 to 8 carbon atoms; k1 and k2 represent an integer of 0 to 4; m1 and m2 represent an integer of 0 to 6; R⁶ is OR⁷; R⁷ represents an organic group having 1 to 30 carbon atoms; n1 and n2 represent an integer of 0 or larger; and n1+2×n2 represents a valence depending on the type of L; and L represents Al

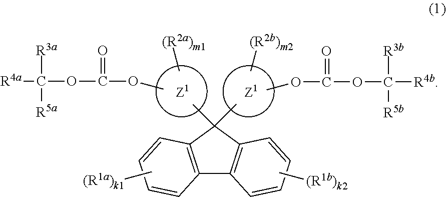

### RELATED APPLICATION

This application is claims priority to Japanese Patent Application 2021-030463, filed Feb. 36 2021, the entire content of which is incorporated herein by reference.

### BACKGROUND OF THE INVENTION

### Field of the Invention

The present invention relates to a metal oxide film-forming composition, a method for producing a metal oxide film using the composition, a tertiary alkyloxycarbonyl group-modified bisnaphthol fluorene compound, and a method for producing the compound.

### Related Art

High refractive index materials are used in formation of optical components. As the high refractive index material, for example, materials obtained by dispersing metal oxide particles such as titanium oxide and zirconium oxide in an organic component are used. As such a high refractive index material, a composition containing metal oxide particles and a fluorene compound having a specific structure including a hydrolyzable silyl group in which a benzene ring is bonded to fluorene has been disclosed (see Patent Document 1). Since the composition in Patent Document 1 contains the fluorene compound having the specific structure including the hydrolyzable silyl group in which the benzene ring is bonded to the fluorene, the composition has a high refractive index and is excellent in a metal oxide particle dispersibility.

Patent Document 1: Japanese Unexamined Patent Application, Publication No. 2012-233142

### SUMMARY OF THE INVENTION

From investigation by the present inventors, it has found that conventional metal oxide film-forming compositions containing metal oxide particles are poor in dispersion stability in some cases, and that metal oxide films obtained by heating the metal oxide film-forming compositions are poor in any of refractive index, surface smoothness, and heat resistance.

The present invention has been made in view of such conventional circumstances, and an object of the present invention is to provide a metal oxide film-forming composition excellent in dispersion stability and obtains a metal oxide film having excellent refractive index, surface smoothness, and heat resistance after heating, a method for producing the metal oxide film using the composition, a tertiary alkyloxycarbonyl group-modified bisnaphthol fluorene compound, and a method for producing the compound.

As a result of intensive investigations by the present inventors for solving the above problems, it has found that the above problems can be solved by a metal oxide film-forming composition containing: a predetermined tertiary alkyloxycarbonyl group-modified bisnaphthol fluorene compound; at least one metal component selected from the group consisting of a predetermined metal compound, a hydrolysate of the metal compound, a condensate of the metal compound, and a hydrolyzed condensate of the metal compound; and a solvent, and this finding has led to the completion of the invention. Specifically, the present invention provides the followings.

A first aspect of the invention is directed to a metal oxide film-forming composition including:

a tertiary alkyloxycarbonyl group-modified bisnaphthol fluorene compound represented by the following formula (1);
at least one metal component selected from the group consisting of a metal compound represented by the following formula (2), a hydrolysate of the metal compound, a condensate of the metal compound, and a hydrolyzed condensate of the metal compound; and
a solvent.

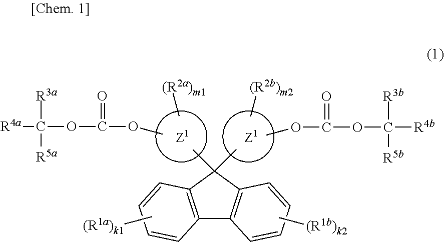

In formula (1),
ring Z¹ represents a naphthalene ring,
R^{1a} and R^{1b} each independently represent a halogen atom, a cyano group, or an alkyl group,
R^{2a} and R^{2b} each independently represent an alkyl group,
R^{3a}, R^{3b}, R^{4a}, R^{4b}, R^{5a}, and R^{5b} each independently represent an alkyl group having 1 to 8 carbon atoms,
k1 and k2 each independently represent an integer of 0 or larger to 4 or smaller, and
m1 and m2 each independently represent an integer of 0 or larger to 6 or smaller.

L(R⁶)ₙ₁(O)ₙ₂ (2)

In formula (2), R⁶ represents a group represented by OR⁷, R⁷ represents an organic group having 1 to 30 carbon atoms, and n1 and n2 each independently represent an integer of 0 or larger, provided that n1+2×n2 is a valence depending on the type of L, and L represents aluminum, gallium, yttrium, titanium, zirconium, hafnium, bismuth, tin, vanadium, or tantalum.

A second aspect of the present invention is directed to a method for producing a metal oxide film, including: a coating film-forming step of forming a coating film composed of the metal oxide film-forming composition according to the first aspect; and a heating step of heating the coating film.

A third aspect of the present invention is directed to a tertiary alkyloxycarbonyl group-modified bisnaphthol fluorene compound represented by the above formula (1).

A fourth aspect of the present invention is directed to a method for producing the tertiary alkyloxycarbonyl group-modified bisnaphthol fluorene compound represented by the above formula (1), including: a step of reacting a bisnaphthol fluorene compound represented by the following formula (3) with a di(tertiary alkyl) dicarbonate compound represented by the following formula (4) in the presence of 25 ppm or less of metal.

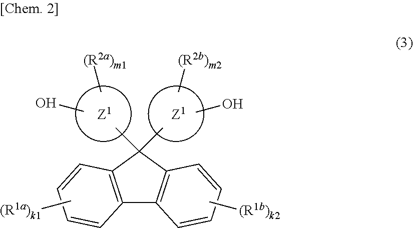

In formula (3), rings Z¹, R^{1a}, R^{1b}, R^{2a}, R^{2b}, k1, k2, m1, and m2 are as defined above.

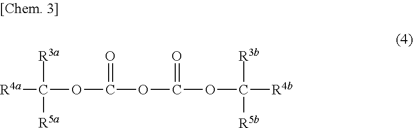

wherein, in formula (4), R^{3a}, R^{3b}, R^{4a}, R^{4b}, R^{5a}, and R^{5b} are as defined above.

According to the present invention, it is possible to provide a metal oxide film-forming composition excellent in dispersion stability and obtains a metal oxide film having excellent refractive index, surface smoothness, and heat resistance after heating, and a method for producing a metal oxide film using the composition, a tertiary alkyloxycarbonyl group-modified bisnaphthol fluorene compound, and a method for producing the compound.

### DETAILED DESCRIPTION OF THE INVENTION

### <Metal Oxide Film-Forming Composition>

The metal oxide film-forming composition according to the present invention contains: the tertiary alkyloxycarbonyl group-modified bisnaphthol fluorene compound represented by the above formula (1); at least one metal component selected from the group consisting of the metal compound represented by the above formula (2), a hydrolysate of the metal compound, a condensate of the metal compound, and a hydrolyzed condensate of the metal compound; and a solvent. The metal oxide film-forming composition according to the present invention is excellent in dispersion stability and can obtain a metal oxide film having excellent refractive index, surface smoothness, and heat resistance after heating.

### [Tertiary Alkyloxycarbonyl Group-Modified Bisnaphthol Fluorene Compound Represented by Formula (1)]

The metal oxide film-forming composition contains the tertiary alkyloxycarbonyl group-modified bisnaphthol fluorene compound represented by the above formula (1). The tertiary alkyloxycarbonyl group-modified bisnaphthol fluorene compound may be used alone or in combination of two or more types thereof.

Specific examples of the halogen atom as R^{1a} and R^{1b} in the above formula (1) include chlorine atom, fluorine atom, bromine atom, and iodine atom. In the above formula (1), the alkyl group as R^{1a} and R^{1b} may be linear or branched chain, and examples of the alkyl group include alkyl groups having 1 or more to 6 or less carbon atoms, such as a methyl group, an ethyl group, a propyl group, an isopropyl group, a butyl group, and a tert-butyl group. R^{1a} and R^{1b} may be the same as or different from each other. When k1 is an integer of 2 or larger, two or more groups R^{1a} may be the same as or different from each other, and when k2 is an integer of 2 or larger, two or more groups R^{1b} may be the same as or different from each other. k1 and k2 are each independently an integer of 0 or larger to 4 or smaller, preferably 0 or 1, more preferably 0. k1 and k2 may be the same as or different from each other.

In the above formula (1), the alkyl group as R^{2a} and R^{2b} may be linear or branched chain, and examples of the alkyl group include alkyl groups having 1 or more to 18 or less carbon atoms, such as a methyl group, an ethyl group, a propyl group, an isopropyl group, a butyl group, a tert-butyl group, an n-pentyl group, an isopentyl group, a sec-pentyl group, a tert-pentyl group, an n-hexyl group, an isohexyl group, a sec-hexyl group, and a tert-hexyl group, preferably an alkyl group having 1 or more to 8 or less carbon atoms, more preferably an alkyl group having 1 or more to 6 or less carbon atoms. R^{2a} and R^{2b} may be the same as or different from each other. When m1 is 2, two groups R^{2a} may be the same as or different from each other, and when m2 is 2, two groups R^{2b} may be the same as or different from each other. m1 and m2 are each independently an integer of 0 or larger to 6 or smaller, preferably an integer of 0 or larger to 3 or smaller, more preferably 0 or 1. m1 and m2 may be the same as or different from each other.

In the above formula (1), examples of the alkyl groups having 1 to 8 carbon atoms represented by R^{3a}, R^{3b}, R^{4a}, R^{4b}, R^{5a} and R^{5b} include, but are not particularly limited to, a methyl group, an ethyl group, a propyl group, an isopropyl group, a butyl group, a tert-butyl group, an n-pentyl group, an isopentyl group, a sec-pentyl group, a tert-pentyl group, an n-hexyl group, an isohexyl group, a sec-hexyl group, a tert-hexyl group, an n-heptyl group, an n-octyl group, and the like. In terms of synthesis easiness, stability, and the like, an alkyl group having 1 or more to 6 or less carbon atoms, such as a methyl group, an ethyl group, a propyl group, an isopropyl group, a butyl group, a tert-butyl group, an n-pentyl group, an isopentyl group, a sec-pentyl group, a tert-pentyl group, an n-hexyl group, an isohexyl group, a sec-hexyl group, and a tert-hexyl group is preferable, an alkyl group having 1 or more to 3 or less carbon atoms, such as a methyl group, an ethyl group, a propyl group, an isopropyl group is more preferable, and a methyl group is even more preferable.

Examples of the tertiary alkyloxycarbonyl group-modified bisnaphthol fluorene compound represented by formula (1) include a tertiary alkyloxycarbonyl group-modified bisnaphthol fluorene compound represented by the following formula (1-1), and the like. Note that, in formula (1-1), R^{2a}, R^{2b}, —O—CO—O—C(R^{3a})(R^{4a})(R^{5a}), and —O—CO—O—C(R^{3b})(R^{4b})(R^{5b}) bonded to a naphthalene ring is bonded to a 6-membered ring not bonded to a fluorene ring among two 6-membered rings constituting the naphthalene ring.

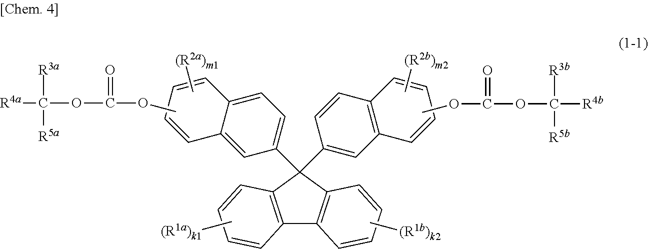

wherein R^{1a}, R^{1b}, R^{2a}, R^{2b}, R^{3a}, R^{3b}, R^{4a}, R^{4b}, R^{5a}, R^{5b}, k1, k2, m1, and m2 are as described above.

Specific examples of the tertiary alkyloxycarbonyl group-modified bisnaphthol fluorene compound represented by formula (1) are as below, but are not limited to the followings.

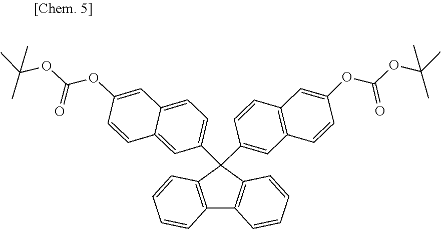 [SMILES: CC(C)(C)OC(=O)Oc1ccc2cc(C3(c4ccc5cc(OC(=O)OC(C)(C)C)ccc5c4)c4ccccc4-c4ccccc43)ccc2c1]

The usage amount of the tertiary alkyloxycarbonyl group-modified bisnaphthol fluorene compound represented by the above formula (1) is not particularly limited, and is, for example, 30 to 70% by mass, preferably 40 to 60% by mass, based on the total amount of components other than the solvent in the metal oxide film-forming composition. If the usage amount of the tertiary alkyloxycarbonyl group-modified bisnaphthol fluorene compound is within the above range, the surface smoothness of the obtained metal oxide film is easily improved.

### (Method for Producing Tertiary Alkyloxycarbonyl Group-Modified Bisnaphthol Fluorene Compound Represented by Formula (1))

The tertiary alkyloxycarbonyl group-modified bisnaphthol fluorene compound represented by the above formula (1) can be produced by reacting a hydroxy group-containing aromatic hydrocarbon ring-modified fluorene compound represented by the following formula (3) with a di(tertiary alkyl) dicarbonate compound represented by the following formula (4) in the presence of 25 ppm or less (by mass, the same shall apply hereinafter) of metal. This reaction may be performed in the presence of a base (e.g., an organic base such as triethylamine, pyridine, and N,N-dimethyl-4-aminopyridine) in a solvent (e.g., an alkyl halide-based solvent such as dichloromethane, an ether-based solvent such as tetrahydrofuran (THF), an alcohol-based solvent such as methanol).

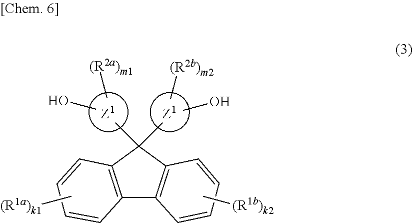

wherein Z¹, R^{1a}, R^{1b}, R^{2a}, R^{2b}, k1, k2, m1, and m2 are as described above.

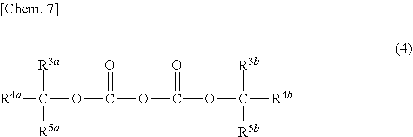

wherein R^{3a}, R^{3b}, R^{4a}, R^{4b}, R^{5a}, and R^{5b} are as described above.

When the reaction of the bisnaphthol fluorene compound represented by the above formula (3) with the di (tertiary alkyl) dicarbonate compound represented by the above formula (4) is performed in the presence of 25 ppm or less of metal, the reaction can be easily advanced with satisfaction, and a sufficient amount of tertiary alkyloxycarbonyl group-modified bisnaphthol fluorene compound represented by the above formula (1) can be easily obtained. From the viewpoint of a yield of the tertiary alkyloxycarbonyl group-modified bisnaphthol fluorene compound, the amount of the metal is preferably 23 ppm or less, more preferably 21 ppm or less. Examples of the metal include, but are not particularly limited to, an alkali metal such as sodium and potassium, and an alkaline earth metal such as magnesium and calcium.

For performing the aforementioned reaction in the presence of 25 ppm or less of metal, it is preferable to use purified products for the hydroxy group-containing aromatic hydrocarbon ring-modified fluorene compound represented by the above formula (3), the di(tertiary alkyl) dicarbonate compound represented by the above formula (4), a base, and a solvent. Examples of the purification method include, but are not limited to, recrystallization, reprecipitation, distillation, and the like. In particular, examples of the purification method for the hydroxy group-containing aromatic hydrocarbon ring-modified fluorene compound represented by the above formula (3) include a purification method including a step of removing impurities from crude products to obtain a purified product (see Japanese Unexamined Patent Application, Publication No. 2016-069289), a purification method including a step of purifying crude products by distillation under vacuum, sublimation purification, recrystallization from an organic solvent solution, washing with an organic solvent, or the like to obtain a semi-purified product, and obtaining a purified product from the semi-purified product by treatment with an adsorbent in an organic solvent, or the like (see Japanese Unexamined Patent Application, Publication No. 2017-171827), and the like.

### [Metal Component]

The metal oxide film-forming composition contains at least one metal component selected from the group consisting of the metal compound represented by the above formula (2), a hydrolysate of the metal compound, a condensate of the metal compound, and a hydrolyzed condensate of the metal compound. The metal component may be used alone or in combination of two or more types thereof.

Metal Compound Represented by Formula (2)

In the above formula (2), when n1 represents an integer of 2 or larger, the plurality of groups R⁶ may be the same as or different from each other.

In the above formula (2), examples of the organic group having 1 to 30 carbon atoms represented by R⁷ include, but are not particularly limited to, an alkyl group having 1 to 30 carbon atoms, a cycloalkyl group having 3 to 30 carbon atoms, an alkenyl group having 2 to 30 carbon atoms, an aryl group having 6 to 30 carbon atoms, and an alkoxyalkyl group having 2 to 30 carbon atoms.

Examples of the alkyl group having 1 to 30 carbon atoms include, but are not particularly limited to, a methyl group, an ethyl group, a propyl group, an isopropyl group, a butyl group, a tert-butyl group, an n-pentyl group, an isopentyl group, a sec-pentyl group, a tert-pentyl group, an n-hexyl group, an isohexyl group, a sec-hexyl group, a tert-hexyl group, an n-heptyl group, an n-octyl group, an n-decyl group, an n-dodecyl group, an n-octadecyl group, an n-icosyl group, and the like. In terms of synthesis easiness, stability, and the like, an alkyl group having 1 or more to 6 or less carbon atoms such as a methyl group, an ethyl group, a propyl group, an isopropyl group, a butyl group, a tert-butyl group, an n-pentyl group, an isopentyl group, a sec-pentyl group, a tert-pentyl group, an n-hexyl group, an isohexyl group, a sec-hexyl group, and a tert-hexyl group is preferable.

Examples of the cycloalkyl group having 3 to 30 carbon atoms include, but are not particularly limited to, a cyclopropyl group, a cyclobutyl group, a cyclopentyl group, a cyclohexyl group, a cycloheptyl group, a cyclooctyl group, a cyclodecyl group, a cyclododecyl group, a cyclooctadecyl group, a cycloicosyl group, and the like. In terms of synthesis easiness, stability, and the like, a cycloalkyl group having 3 or more to 6 or less carbon atoms, such as a cyclopropyl group, a cyclobutyl group, a cyclopentyl group, and a cyclohexyl group is preferable.

Examples of the alkenyl group having 2 to 30 carbon atoms include, but are not particularly limited to, a vinyl group, an allyl group, and the like. In terms of synthesis easiness, stability, and the like, the allyl group is preferable.

Examples of the aryl group having 6 to 30 carbon atoms include, but are not particularly limited to, a phenyl group, a naphthyl group, and the like. In terms of synthesis easiness, stability, and the like, the phenyl group is preferable.

Examples of the alkoxyalkyl group having 2 to 30 carbon atoms include, but are not particularly limited to, a methoxymethyl group, a methoxyethyl group, an ethoxymethyl group, an ethoxyethyl group, and the like. In terms of synthesis easiness, stability, and the like, the methoxyethyl group and the ethoxyethyl group are preferable.

In the above formula (2), for example, R⁶ is represented by OR⁷, and examples of R⁷ include the alkyl group having 1 to 30 carbon atoms, the cycloalkyl group having 3 to 30 carbon atoms, the alkenyl group having 2 to 30 carbon atoms, the aryl group having 6 to 30 carbon atoms, the alkoxyalkyl group having 2 to 30 carbon atoms, as well as an alkylacetoacetate group having 5 to 30 carbon atoms, a 2,4-pentanedionato group (i.e. acetylacetonato group), a 2,2,6,6-tetramethyl-3,5-heptanedionato group.

Examples of the alkyl acetoacetate group having 5 to 30 carbon atoms include, but are not limited to, a methylacetoacetate group, an ethylacetoacetate group, and the like. In terms of synthesis easiness, stability, and the like, the ethylacetoacetate group is preferable.

When L represents aluminum, examples of the metal compound represented by the above formula (2) include aluminum methoxide, aluminum ethoxide, aluminum propoxide, aluminum isopropoxide, aluminum butoxide, aluminum amyloxide, aluminum hexyloxide, aluminum cyclopentoxide, aluminum cyclohexyloxide, aluminum allyloxide, aluminum phenoxide, aluminum methoxyethoxide, aluminum ethoxyethoxide, aluminum dipropoxyethyl acetoacetate, aluminum dibutoxyethyl acetoacetate, aluminum propoxybis ethylacetoacetate, aluminum butoxybis ethylacetoacetate, aluminum 2,4-pentanedionate, aluminum 2,2,6,6-tetramethyl-3,5-heptanedionate, and the like.

When L represents gallium, examples of the metal compound represented by the above formula (2) include gallium methoxide, gallium ethoxide, gallium propoxide, gallium isopropoxide, gallium butoxide, gallium amiloxide, gallium hexyloxide, gallium cyclopentoxide, gallium cyclohexyloxide, gallium allyloxide, gallium phenoxide, gallium methoxyethoxide, gallium ethoxyethoxide, gallium dipropoxyethyl acetoacetate, gallium dibutoxyethyl acetoacetate, gallium propoxybis ethylacetoacetate, gallium butoxybis ethylacetoacetate, gallium 2,4-pentanedionate, gallium 2,2,6,6-tetramethyl-3,5-heptanedionate, and the like.

When L represents yttrium, examples of the metal compound represented by the above formula (2) include yttrium methoxide, yttrium ethoxide, yttrium propoxide, yttrium isopropoxide, yttrium butoxide, yttrium amyloxide, yttrium hexyloxide, yttrium cyclopentoxide, yttrium cyclohexyloxide, yttrium allyloxide, yttrium phenoxide, yttrium methoxyethoxide, yttrium ethoxyethoxide, yttrium dipropoxyethyl acetoacetate, yttrium dibutoxyethyl acetoacetate, yttrium propoxybis ethylacetoacetate, yttrium butoxybis ethylacetoacetate, yttrium 2,4-pentanedionate, yttrium 2,2,6,6-tetramethyl-3,5-heptanedionate, and the like.

When L represents titanium, examples of the metal compound represented by the above formula (2) include titanium methoxide, titanium ethoxide, titanium propoxide, titanium isopropoxide, titanium butoxide, titanium amyloxide, titanium hexyloxide, titanium cyclopentoxide, titanium cyclohexyloxide, titanium allyloxide, titanium phenoxide, titanium methoxyethoxide, titanium ethoxyethoxide, titanium dipropoxybis ethylacetoacetate, titanium dibutoxybis ethylacetoacetate, titanium dipropoxybis 2,4-pentanedionate, bis(2,4-pentanedionato) titanium oxide, titanium dibutoxybis 2,4-pentanedionate, and the like.

When L represents zirconium, examples of the metal compound represented by the above formula (2) include methoxy zirconium, ethoxy zirconium, propoxy zirconium, isopropoxy zirconium, butoxy zirconium, phenoxy zirconium, zirconium dibutoxide bis(2,4-pentanedionate), bis(2,4-pentanedionato) zirconium oxide, zirconium dipropoxide bis(2,2,6,6-tetramethyl-3,5-heptanedionate) and the like.

When L represents hafnium, examples of the metal compound represented by the above formula (2) include hafnium methoxide, hafnium ethoxide, hafnium propoxide, hafnium isopropoxide, hafnium butoxide, hafnium amyloxide, hafnium hexyloxide, hafnium cyclopentoxide, hafnium cyclohexyloxide, hafnium allyloxide, hafnium phenoxide, hafnium methoxyethoxide, hafnium ethoxyethoxide, hafnium dipropoxybis ethylacetoacetate, hafnium dibutoxybis ethylacetoacetate, hafnium dipropoxybis 2,4-pentanedionate, hafnium dibutoxybis 2,4-pentanedionate, and the like.

When L represents bismuth, examples of the metal compound represented by the above formula (2) include methoxy bismuth, ethoxy bismuth, propoxy bismuth, isopropoxy bismuth, butoxy bismuth, phenoxy bismuth, and the like.

When L represents tin, examples of the metal compound represented by the above formula (2) include methoxy tin, ethoxy tin, propoxy tin, isopropoxy tin, butoxy tin, phenoxy tin, methoxyethoxy tin, ethoxyethoxy tin, tin 2,4-pentanedionate, tin 2,2,6,6-tetramethyl-3,5-heptanedionate, and the like.

When L represents vanadium, examples of the metal compound represented by the above formula (2) include vanadium oxide bis(2,4-pentanedionate), vanadium 2,4-pentanedionate, vanadium tributoxide oxide, vanadium tripropoxide oxide, and the like.

When L represents tantalum, examples of the metal compound represented by the above formula (2) include methoxy tantalum, ethoxy tantalum, propoxy tantalum, isopropoxy tantalum, butoxy tantalum, phenoxy tantalum, and the like.

Hydrolysate of Metal Compound, Condensate of Metal Compound, and Hydrolyzed Condensate of Metal Compound

The metal compound represented by the above formula (2) can be hydrolyzed, condensed, or hydrolytically condensed in the absence of catalyst and in the presence of an acid or alkali catalyst to produce a hydrolysate of the metal compound, a condensate of the metal compound, or a hydrolyzed condensate of the metal compound. At this time, one or more compounds selected from the group consisting of an inorganic acid, an aliphatic sulfonic acid, an aromatic sulfonic acid, an aliphatic carboxylic acid, and an aromatic carboxylic acid can be used as the acid catalyst. Note that the hydrolysate of the metal compound, the condensate of the metal compound, and the hydrolyzed condensate of the metal compound are hereinafter also referred to as “metal oxide-containing compounds”.

Examples of the acid catalyst used in this process include hydrofluoric acid, hydrochloric acid, hydrobromic acid, sulphuric acid, nitric acid, perchloric acid, phosphoric acid, methanesulfonic acid, benzenesulphonic acid, toluenesulphonic acid, formic acid, acetic acid, propionic acid, oxalic acid, malonic acid, maleic acid, fumaric acid, benzoic acid, and the like. A usage amount of the catalyst is preferably 10⁻⁶ to 10 moles, more preferably 10⁻⁵ to 5 moles, even more preferably 10⁻⁴ to 1 mole, based on 1 mole of monomer.

The metal compound may be produced by hydrolytic condensation in the presence of an alkali catalyst. Examples of the alkali catalyst used in this process include methylamine, ethylamine, propylamine, butylamine, ethylenediamine, hexamethylenediamine, dimethylamine, diethylamine, ethylmethylamine, trimethylamine, triethylamine, tripropylamine, tributylamine, cyclohexylamine, dicyclohexylamine, monoethanolamine, diethanolamine, dimethyl monoethanolamine, monomethyl diethanolamine, triethanolamine, diazabicyclooctane, diazabicyclo cyclononene, diazabicyclo undecene, hexamethylenetetramine, aniline, N,N-dimethylaniline, pyridine, N,N-dimethylethanolamine, N,N-diethylethanolamine, N-(β-aminoethyl)ethanolamine, N-methylethanolamine, N-methyldiethanolamine, N-ethylethanolamine, N-n-butylethanolamine, N-n-butyldiethanolamine, N-tert-butylethanolamine, N-tert-butyldiethanolamine, N,N-dimethylaminopyridine, pyrrole, piperazine, pyrrolidine, piperidine, picoline, tetramethylammonium hydroxide, choline hydroxide, tetrapropylammonium hydroxide, tetrabutylammonium hydroxide, ammonia, lithium hydroxide, sodium hydroxide, potassium hydroxide, barium hydroxide, calcium hydroxide, and the like. A usage amount of the catalyst is preferably 10⁻⁶ to 10 moles, more preferably 10⁻⁵ to 5 moles, even more preferably 10⁻⁴ to 1 mole, based on 1 mole of metal compound monomer.

When the metal oxide-containing compound is obtained from the metal compound by hydrolysis, condensation, or hydrolytic condensation in this way, the amount of water is preferably 0.01 to 100 moles, more preferably 0.05 to 50 moles, even more preferably 0.1 to 30 moles per 1 mole of hydrolysable substituent bonded to the metal compound. The water addition amount of 100 moles or less will not excessively enlarge a device used for the reaction, and therefore the reaction is economical.

In a method of the operation, the metal compound is added to the catalyst aqueous solution to initiate hydrolysis, condensation, or hydrolytic condensation. At this time, an organic solvent may be added to the catalyst aqueous solution, or the metal compound may be previously diluted with an organic solvent, or both the former and the latter may be conducted. The reaction temperature is preferably 0 to 100° C., more preferably 5 to 80° C. A preferable method is to maintain the temperature at 5 to 80° C. during the dripping of the metal compound and then to mature the metal compound at 20 to 80° C.

In another operation for reaction, water or a hydrous organic solvent is added to the metal compound or a metal compound organic solution to initiate hydrolysis, condensation, or hydrolytic condensation. At this time, the catalyst may be added to the metal compound or the metal compound organic solution, or added to water or a hydrous organic solvent. The reaction temperature is preferably 0 to 100° C., more preferably 10 to 80° C. A preferable method is to heat the metal compound to 10 to 50° C. during the dripping of water, and then to raise the temperature to 20 to 80° C. for maturing.

Preferable examples of the organic solvent capable of being added to the catalyst aqueous solution or the organic solvent capable of diluting the metal compound include methanol, ethanol, 1-propanol, 2-propanol, 1-butanol, 2-butanol, 2-methyl-1-propanol, acetone, acetonitrile, tetrahydrofuran, toluene, hexane, ethyl acetate, cyclohexanone, methylamylketone, butanediol monomethyl ether, propylene glycol monomethyl ether, ethylene glycol monomethyl ether, butanediol monoethyl ether, propylene glycol monoethyl ether, ethylene glycol monoethyl ether, propylene glycol dimethyl ether, diethylene glycol dimethyl ether, propylene glycol monomethyl ether acetate, propylene glycol monoethyl ether acetate, ethyl pyruvate, butyl acetate, methyl 3-methoxypropionate, ethyl 3-ethoxypropionate, tert-butyl acetate, t-butyl propionate, propylene glycol mono-t-butyl ether acetate, γ-butyrolactone, acetyl acetone, methyl acetoacetate, ethyl acetoacetate, propyl acetoacetate, butyl acetoacetate, methyl pivaloyl acetate, methyl isobutyroyl acetate, methyl caproyl acetate, methyl lauroyl acetate, 1,2-ethanediol, 1,2-propanediol, 1,2-butanediol, 1,2-pentanediol, 2,3-butanediol, 2,3-pentanediol, glycerin, diethylene glycol, hexylene glycol, a mixtures of two or more thereof, and the like.

A usage amount of the organic solvent is preferably 0 to 1,000 ml, particularly preferably 0 to 500 ml based on 1 mole of the metal compound. The organic solvent usage amount of 1,000 ml or less will not excessively enlarge a reaction container, and therefore the reaction is economical.

If necessary, the catalyst is then subjected to a neutralization reaction, and alcohols generated in the hydrolysis, condensation, or hydrolytic condensation are removed under reduced pressure to obtain an aqueous solution of the reaction mixture. At this time, an amount of an acid or an alkali usable for neutralization is preferably 0.1 to 2 equivalents based on the acid or alkali used for the catalyst. The acid or alkali may be any substance as long as the aqueous solution of the reaction mixture is neutralized.

It is preferable to then remove by-products such as alcohols generated in the hydrolysis, condensation, or hydrolytic condensation from the reaction mixture. At this time, the temperature for heating the reaction mixture is preferably 0 to 100° C., more preferably 10 to 90° C., even more preferably 15 to 80° C. depending on the types of the added organic solvent, the alcohol generated in the reaction, and the like. At this time, the degree of pressure reduction depends on the types of the organic solvent and alcohol to be removed, an exhauster, a condenser, and the heating temperature, and the pressure is preferably atmospheric pressure or lower, more preferably 80 kPa or lower in absolute pressure, even more preferably 50 kPa or lower in absolute pressure. In this process, although it is difficult to know exactly the amount of the alcohol to be removed, it is desirable that approximately 80% by mass or more of the generated alcohol and the like is removed.

Preferable examples of the final solvent to be added to the metal oxide-containing compound solution include butanediol monomethyl ether, propylene glycol monomethyl ether, ethylene glycol monomethyl ether, butanediol monoethyl ether, propylene glycol monoethyl ether, ethylene glycol monoethyl ether, butanediol monopropyl ether, propylene glycol monopropyl ether, ethylene glycol monopropyl ether, ethylene glycol monobutyl ether, diethylene glycol monomethyl ether, diethylene glycol monoethyl ether, diethylene glycol monopropyl ether, diethylene glycol monobutyl ether, propylene glycol monobutyl ether, 1-butanol, 2-butanol, 2-methyl-1-propanol, 4-methyl-2-pentanol, acetone, tetrahydrofuran, toluene, hexane, ethyl acetate, cyclohexanone, methyl amyl ketone, propylene glycol dimethyl ether, diethylene glycol dimethyl ether, diamyl ether, propylene glycol monomethyl ether acetate, propylene glycol monoethyl ether acetate, ethyl pyruvate, butyl acetate, methyl 3-methoxypropionate, ethyl 3-ethoxypropionate, t-butyl acetate, t-butyl propionate, propylene glycol mono-t-butyl ether acetate, γ-butyrolactone, methyl isobutyl ketone, cyclopentyl methyl ether, and the like.

A molecular weight of the obtained metal oxide-containing compound can be adjusted not only by selecting the metal compound but also by controlling the reaction conditions during hydrolysis, condensation, or hydrolytic condensation. If a mass-average molecular weight is 100,000 or less, foreign matters or coating spots are not generated, and therefore the mass-average molecular weight is preferably 100,000 or less, more preferably 200 to 50,000, even more preferably 300 to 30,000. Note that data on the mass-average molecular weight presents a molecular weight in terms of polystyrene, determined using polystyrene as a standard substance by gel permeation chromatography (GPC) using RI as a detector and tetrahydrofuran as an eluting solvent.

The usage amount of the metal component is not particularly limited and is, for example, 30 to 70% by mass, preferably 40 to 60% by mass, based on the total amount of components other than the solvent in the metal oxide film-forming composition. If the usage amount of the metal component is within the above range, the surface smoothness of the obtained metal oxide film is easily improved.

### [Solvent]

The metal oxide film-forming composition according to the present invention contains a solvent for the purpose of adjusting the coatability and viscosity. As the solvent, an organic solvent is typically used. There is no particular limitation on types of the organic solvent as long as it can uniformly dissolve or disperse components contained in the metal oxide film-forming composition.

Suitable examples of the organic solvent usable as the solvent include (poly)alkylene glycol monoalkyl ethers such as ethylene glycol monomethyl ether, ethylene glycol monoethyl ether, ethylene glycol n-propyl ether, ethylene glycol mono-n-butyl ether, diethylene glycol monomethyl ether, diethylene glycol monoethyl ether, diethylene glycol mono-n-propyl ether, diethylene glycol mono-n-butyl ether, triethylene glycol monomethyl ether, triethylene glycol monoethyl ether, propylene glycol monomethyl ether, propylene glycol monoethyl ether, propylene glycol mono-n-propyl ether, propylene glycol mono-n-butyl ether, dipropylene glycol monomethyl ether, dipropylene glycol monoethyl ether, dipropylene glycol mono-n-propyl ether, dipropylene glycol mono-n-butyl ether, tripropylene glycol monomethyl ether, and tripropylene glycol monoethyl ether; (poly)alkylene glycol monoalkyl ether acetates such as ethylene glycol monomethyl ether acetate, ethylene glycol monoethyl ether acetate, diethylene glycol monomethyl ether acetate, diethylene glycol monoethyl ether acetate, propylene glycol monomethyl ether acetate, and propylene glycol monoethyl ether acetate; other ethers such as diethylene glycol dimethyl ether, diethylene glycol methyl ethyl ether, diethylene glycol diethyl ether, and tetrahydrofuran; ketones such as methyl ethyl ketone, cyclohexanone, 2-heptanone, 3-heptanone, and acetylacetone; lactic acid alkyl esters such as methyl 2-hydroxypropionate and ethyl 2-hydroxypropionate; other esters such as ethyl 2-hydroxy-2-methylpropionate, methyl 3-methoxypropionate, ethyl 3-methoxypropionate, methyl 3-ethoxypropionate, ethyl 3-ethoxypropionate, ethyl ethoxyacetate, ethyl hydroxyacetate, methyl 2-hydroxy-3-methylbutanoate, 3-methyl-3-methoxybutyl acetate, 3-methyl-3-methoxybutyl propionate, ethyl acetate, n-propyl acetate, isopropyl acetate, n-butyl acetate, isobutyl acetate, n-pentyl formate, isopentyl acetate, n-butyl propionate, ethyl butyrate, n-propyl butyrate, isopropyl butyrate, n-butyl butyrate, methyl pyruvate, ethyl pyruvate, n-propyl pyruvate, methyl acetoacetate, ethyl acetoacetate, ethyl 2-oxobutanoate, and cyclohexanol acetate; aromatic hydrocarbons such as toluene and xylene; and amides such as N-methylpyrrolidone, N,N-dimethylformamide, and N,N-dimethylacetamide. These organic solvents can be used alone or in combination of two or more types thereof. For example, acetylacetone may be used in combination with other solvents (e.g., propylene glycol monomethyl ether acetate or cyclohexanol acetate) because the dispersion stability of the obtained metal oxide film-forming composition is further easily improved.

There is no particular limitation on the usage amount of the solvent in the metal oxide film-forming composition according to the present invention. In view of the coatability and the like of the metal oxide film-forming composition, the usage amount of the solvent is, for example, 30 to 99.9% by mass, preferably 50 to 98% by mass based on the entire metal oxide film-forming composition. When acetylacetone is used in combination with another solvent as described above, the content of acetylacetone is preferably 1.5 or more times mole, more preferably 2 or more times mole as much as the metal compound in the metal oxide film-forming composition in terms of the stability of the metal oxide film-forming composition. The upper limit of the content of the acetylacetone only needs to be appropriately adjusted, and it is preferable that the content of the acetylacetone is preferably, for example, 50% by mass or less based on the entire solvent contained in the metal oxide film-forming composition in terms of solubility of the tertiary alkyloxycarbonyl group-modified bisnaphthol fluorene compound represented by the above formula (1).

### [Other Components]

The metal oxide film-forming composition according to the present invention can optionally contain additives such as surfactants (surface conditioner), dispersants, thermal polymerization inhibitors, defoamers, silane coupling agents, colorants (pigments, dyes), inorganic fillers, organic fillers, crosslinking agents, and oxygen generating agents. For all of the additives, a conventionally known additive can be used. Examples of the surfactant include anionic, cationic, and nonionic compounds. Examples of the thermal polymerization inhibitor include hydroquinone, hydroquinone monoethyl ether, and the like. Examples of the defoamer include silicone-based compounds, fluorine-based compounds, and the like.

Examples of the method for producing the metal oxide film-forming composition according to the present invention include, but are not particularly limited to, a method of homogeneously mixing the tertiary alkyloxycarbonyl group-modified bisnaphthol fluorene compound represented by the above formula (1), the metal compound, a solvent, and optionally other components.

### <Method for Producing Metal Oxide Film>

The method for producing a metal oxide film according to the present invention includes a coating film forming step of forming a coating film composed of the metal oxide film-forming composition according to the present invention, and a heating step of heating the coating film.

For example, the metal oxide film-forming composition is applied onto a substrate such as a semiconductor substrate to form the coating film. Examples of the coating method include methods using a contact transfer-type coating apparatus such as a roll coater, a reverse coater, and a bar coater, or a non-contacting-type coating apparatus such as a spinner (rotary coating apparatus, spin coater), a dip coater, a spray coater, a slit coater, and a curtain flow coater. After adjusting the viscosity of the metal oxide film-forming composition in an appropriate range, the metal oxide film-forming composition may be applied by a printing method such as an inkjet method and a screen printing method to form a coating film with a desired pattern shape.

The substrate preferably contains a metal film, a metal carbide film, a metal oxide film, a metal nitride film, or a metal oxynitride film. The metal constituting the substrate preferably contains silicon, titanium, tungsten, hafnium, zirconium, chromium, germanium, copper, aluminum, indium, gallium, arsenic, palladium, iron, tantalum, iridium, molybdenum or an alloy thereof, and above all, silicon, germanium and gallium are preferable. In addition, the substrate surface may be uneven, and the uneven shape may be formed from a patterned organic material.

Then, as necessary, a volatile component such as a solvent is removed to dry the coating film. Examples of the drying method include, but are not particularly limited to, a method of drying the coating film with a hot plate at a temperature of 80° C. or higher to 120° C. or lower, preferably 90° C. or higher to 110° C. or lower, for 90 seconds or more to 150 seconds or less. Prior to the heating with the hot plate, reduced pressure drying may be conducted using a vacuum drying device (VCD) at room temperature.

After the coating film is formed in this way, the coating film is heated. The temperature for the heating is not particularly limited, and from the viewpoint of the curability of the coating film, the temperature is preferably 130° C. or higher, more preferably 140° C. or higher, even more preferably 145° C. or higher. The upper limit of the temperature only needs to be set as appropriate, and the temperature only needs to be set to, for example, 250° C. or lower, and in terms of heat resistance, preferably 200° C. or lower, more preferably 155° C. or lower. The heating time is typically 30 seconds or more and 150 seconds or less, more preferably 60 seconds or more to 120 seconds. The heating step may be performed at a single heating temperature or may be performed at a plurality of stages of different heating temperatures.

The metal oxide film formed as described above is suitably used, for example, as a metal hard mask or a material for pattern reversal. Preferably, the above metal oxide film has a refractive index of 1.8 or higher at a temperature of 25° C. and a wavelength of 550 nm. Thus, the metal oxide film is suitably used for optical applications requiring a high refractive index. For example, the above metal oxide film is suitably used as a high refractive index film constituting an antireflective film or the like in a display panel such as an organic EL display panel and a liquid crystal display panel. The film thickness of the above metal oxide film is not particularly limited and may be appropriately selected according to the application. The film thickness may be typically 1 nm or larger to 20 μm or smaller, and also 50 nm or larger to 10 μm or smaller.

### Examples

The present invention will be described below in more detail by way of Examples, but the present invention is not limited to these Examples.

### [Preparation of Metal Oxide Film-Forming Composition]

### (Modified Fluorene Compound)

Preparation of Modified Bisnaphthol Fluorene Compound 1-A

A bisnaphthol fluorene represented by the following formula 3-A was reacted with a di-tert-butyl dicarbonate represented by the following formula 4-A in the presence of N,N-dimethyl-4-aminopyridine in dichloromethane. As the bisnaphthol fluorene, the di-tert-butyl dicarbonate, the organic base, and the solvent, purified products obtained by recrystallization, reprecipitation, or distillation were used.

Sodium carbonate was added to the bisnaphthol fluorene before the reaction, and the aforementioned reaction was performed in the presence of 20 ppm (by mass) of sodium to obtain the following modified bisnaphthol fluorene compound 1-A. As a result of measuring a refractive index of the obtained modified bisnaphthol fluorene compound 1-A using a spectroscopic ellipsometer (trade name: M-2000, manufactured by J. A. Woollam Japan) at a temperature of 25° C. and a wavelength of 550 nm, the refractive index was proved to be 1.68.

Potassium carbonate was added to the bisnaphthol fluorene before the reaction, and the aforementioned reaction was performed in the presence of 20 ppm (by mass) of potassium to obtain the following modified bisnaphthol fluorene compound 1-A. As a result of measuring a refractive index of the obtained modified bisnaphthol fluorene compound 1-A in the same way as above, the refractive index was proved to be 1.68.

On the other hand, sodium carbonate was added to the bisnaphthol fluorene before the reaction, and the aforementioned reaction was performed in the presence of 28 ppm (by mass) of potassium, and as a result, the following modified bisnaphthol fluorene compound 1-A was not obtained.

Preparation of Modified Bisnaphthol Fluorene Compound C1-A

A bisnaphthol fluorene represented by the following formula C3-A was reacted with a di-tert-butyl dicarbonate represented by the following formula 4-A in the presence of N,N-dimethyl-4-aminopyridine in dichloromethane to obtain a modified bisnaphthol fluorene compound C1-A described below.

Preparation of Modified Bisnaphthol Fluorene Compound C1-B

35 g of bisphenol fluorene represented by the following formula C3-A was mixed with 350 g of diethyl ether, and 1 g of p-toluenesulphonic acid was added to the obtained mixture. Subsequently, 15 g of ethyl vinyl ether was added to the mixture at 15° C. to 25° C. for 30 minutes. The mixture was further stirred for 30 minutes, then 5 g of triethylamine was added to the mixture, and 50 g of 5% by mass sodium hydroxide aqueous solution was then added, stirred, and allowed to stand, and then liquids were separated. To an obtained organic phase, 100 g of pure water was added, stirred, and allowed to stand, and then liquids were separated. This process was repeated twice. The remaining organic phase was condensed under reduced pressure to obtain 47 g of modified bisphenol fluorene compound C1-B.

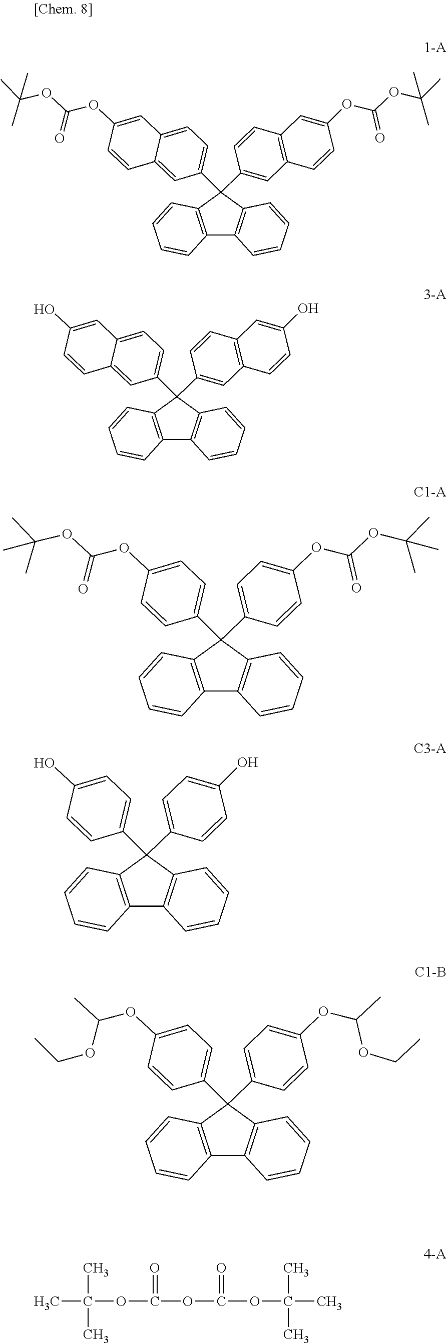

### (Metal Compound)

Ti(OBt)₄: titanium tetrabutoxide
Ti(OiPr)₄: titanium tetraisopropoxide
Ti(acac)₂: bis(2,4-pentanedionato) titanium oxide
Zr(OBt)₄: zirconium tetrabutoxide
Zr(acac)₂: Bis(2,4-pentanedionato) zirconium oxide
Hydrolyzed condensate
   A mixture of 2.7 g of pure water and 50 g isopropyl alcohol was dripped to a mixture of 28.4 g of titanium tetraisopropoxide, 50 g of isopropyl alcohol, and 11.8 g of 2-(butylamino) ethanol. After termination of the dripping, the mixture was stirred for 2 hours to advance hydrolytic condensation, and then refluxed for another 2 hours. To the resulting reaction mixture, 100 g of propylene glycol monomethyl ether acetate (PGMEA) was added, and the mixture was condensed under reduced pressure to obtain 130 g of PGMEA solution of the hydrolyzed condensate. In this solution, a solid concentration of the hydrolyzed condensate was 13.5% by mass.

### (Solvent)

CHXA: Cyclohexanol acetate
Acac: Acetylacetone
PGMEA: Propylene glycol monomethyl ether acetate

A fluorene compound and a metal compound were added to a solvent 1 or a mixture of the solvent 1 and a solvent 2 in types and masses presented in Table 1, stirred, and filtered through a Φ0.2 μm membrane filter to prepare a composition. Note that, in Table 1, the masses of the fluorene compound and the metal compound represent a mass of solid contents.

### [Dispersion Stability]

The composition prepared as described above was visually observed and the dispersion stability of the composition was evaluated in accordance with the following criteria. The results are presented in Table 1.

OK (good): No turbidity was observed within 30 minutes after the preparation.
NG (poor): Turbidity such as cloudiness was observed within 30 minutes after the preparation.

### [Production of Metal Oxide Film]

The composition was dripped onto a 6-inch silicon wafer and spin-coated. Subsequently, the composition was pre-baked using a hot plate at 100° C. for 120 seconds, and post-baked at 150° C. for 90 seconds to obtain a metal oxide film having a thickness of about 60 nm.

### [Refractive Index]

The refractive index of the obtained metal oxide film was measured using a spectroscopic ellipsometer (trade name: M-2000, manufactured by J. A. Woollam Japan) at a temperature of 25° C. and a wavelength of 550 nm and evaluated in accordance with the following criteria. The results are presented in Table 1.

A (good): The refractive index was 1.8 or higher.
B (slightly poor): The refractive index was 1.7 or higher to lower than 1.8.
C (poor): The refractive index was lower than 1.7.

### [Surface Smoothness]

Surface roughness of the obtained metal oxide film was measured as an arithmetic average roughness Ra using a stylus type surface roughness meter (Dektak 150 from ULVAC, Inc.) and evaluated in accordance to the following criteria. The results are presented in Table 1.

OK (good): Ra was 200 Å or lower.
NG (poor): Ra was higher than 200 Å.

### [Heat Resistance]

A mass change of the obtained metal oxide film was measured using a thermogravimetric/differential thermal simultaneous measurement apparatus (trade name: STA 449 Jupiter, manufactured by NETZSCH Japan) at 30° C. to 600° C. and a temperature elevation rate of 10° C./min. The heat resistance of the metal oxide film was evaluated in accordance with the following criteria. The results are presented in Table 1.

OK (good): Temperature in 5% weight loss was 450° C. or higher.
NG (poor): Temperature in 5% weight loss was lower than 450° C.

TABLE 1

| | | | | | Comparative | | | Comparative | | | |
|---|---|---|---|---|---|---|---|---|---|---|---|
| | Examples | | | | Example | Examples | | Examples | | | |
| | 1 | 2 | 3 | 4 | 1 | 5 | 6 | 2 | 3 | 4 | 5 |
| Fluorene | 1-A | 1-A | 1-A | 1-A | C1-A | 1-A | 1-A | C1-A | — | 3-A | C1-B |
| compound | | | | | | | | | | | |
| 0.8 g | | | | | | | | | | | |
| Metal | Ti | Ti | Ti | Ti | Ti | Zr | Zr | Hydrolyzed | Ti | Ti | Hydrolyzed |
| compound | (OBt)₄ | (OBt)₄ | (OiPr)₄ | (acac)₂ | (acac)₂ | (OBt)₄ | (acac)₂ | condensate | (OBt)₄ | (OBt)₄ | condensate |
| 0.8 g | | | | | | | | | | | |
| Solvent 1 | CHXA | CHXA | CHXA | CHXA | PGMEA | CHXA | CHXA | PGMEA | CHXA | CHXA | PGMEA |
| 15.2 g | | | | | | | | | | | |
| Solvent 2 | | Acac | | | | | | | | | |
| 0.4 g | | | | | | | | | | | |
| Dispersion | OK | OK | OK | OK | OK | OK | OK | OK | OK | NG | OK |
| stability | | | | | | | | | | | |
| Refractive | A | A | A | A | B | A | A | B | A | — | C |
| index | | | | | | | | | | | |
| Surface | OK | OK | OK | OK | OK | OK | OK | OK | NG | — | OK |
| smoothness | | | | | | | | | | | |
| Heat | OK | OK | OK | OK | OK | OK | OK | OK | OK | — | NG |
| resistance | | | | | | | | | | | |

As can be seen from Table 1, it was confirmed that, in Examples, the compositions were excellent in dispersion stability and the metal oxide films obtained after heating the compositions were excellent in refractive index, surface smoothness, and heat resistance, whereas, in Comparative Examples, the compositions were poor in dispersion stability, or the metal oxide films were poor in any of refractive index, surface smoothness, and heat resistance.

What is claimed is:

1. A metal oxide film-forming composition comprising:
a tertiary alkyloxycarbonyl group-modified bisnaphthol fluorene compound represented by the following formula (1);
at least one metal component selected from the group consisting of a metal compound represented by the following formula (2), a hydrolysate of the metal compound, a condensate of the metal compound, and a hydrolyzed condensate of the metal compound; and
a solvent, 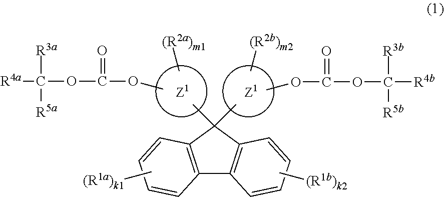
wherein, in formula (1),
ring Z¹ represents a naphthalene ring,
R^{1a} and R^{1b} each independently represents a halogen atom, a cyano group, or an alkyl group,
R^{2a} and R^{2b} each independently represents an alkyl group,
R^{3a}, R^{3b}, R^{4a}, R^{4b}, R^{5a}, and R^{5b} each independently represent an alkyl group having 1 to 8 carbon atoms,
k1 and k2 each independently represents an integer of 0 or larger to 4 or smaller, and
m1 and m2 each independently represents an integer of 0 or larger to 6 or smaller,
L(R⁶)ₙ₁(O)ₙ₂ (2)
wherein, in formula (2), R⁶ represents a group represented by OR⁷, R⁷ represents an organic group having 1 to 30 carbon atoms, and n1 and n2 each independently represents an integer of 0 or larger, provided that n1+2×n2 is a valence depending on the type of L, and L represents aluminum, gallium, yttrium, titanium, zirconium, hafnium, bismuth, tin, vanadium, or tantalum.

2. The metal oxide film-forming composition according to claim 1, wherein R^{3a}, R^{3b}, R^{4a}, R^{4b}, R^{5a}, and R^{5b} are each a methyl group.

3. A method for producing a metal oxide film, comprising:
forming a coating film composed of the metal oxide film-forming composition according to claim 1; and
heating the coating film.

4. A tertiary alkyloxycarbonyl group-modified bisnaphthol fluorene compound represented by the following formula (1), 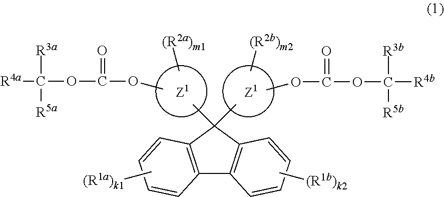
wherein in formula (1),
ring Z¹ represents a naphthalene ring,
R^{1a} and R^{1b} each independently represents a halogen atom, a cyano group, or an alkyl group,
R^{2a} and R^{2b} each independently represents an alkyl group,
R^{3a}, R^{3b}, R^{4a}, R^{4b}, R^{5a}, and R^{5b} each independently represents an alkyl group having 1 to 8 carbon atoms,
k1 and k2 each independently represents an integer of 0 or larger to 4 or smaller, and
m1 and m2 each independently represents an integer of 0 or larger to 6 or smaller.

5. The tertiary alkyloxycarbonyl group-modified bisnaphthol fluorene compound according to claim 4, wherein R^{3a}, R^{3b}, R^{4a}, R^{4b}, R^{5a}, and R^{5b} are each a methyl group.

6. A method for producing a tertiary alkyloxycarbonyl group-modified bisnaphthol fluorene compound represented by the following formula (1), comprising reacting a bisnaphthol fluorene compound represented by the following formula (3) with a di(tertiary alkyl) dicarbonate compound represented by the following formula (4) in the presence of 25 ppm or less of metal, 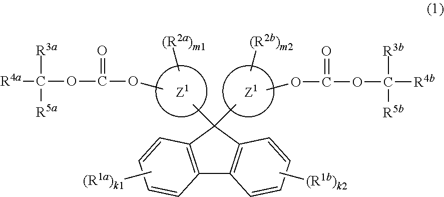
wherein in formula (1),
ring Z¹ represents a naphthalene ring,
R^{1a} and R^{1b} each independently represents a halogen atom, a cyano group, or an alkyl group,
R^{2a} and R^{2b} each independently represents an alkyl group,
R^{3a}, R^{3b}, R^{4a}, R^{4b}, R^{5a}, and R^{5b} each independently represent an alkyl group having 1 to 8 carbon atoms,
k1 and k2 each independently represents an integer of 0 or larger to 4 or smaller, and
m1 and m2 each independently represents an integer of 0 or larger to 6 or smaller, 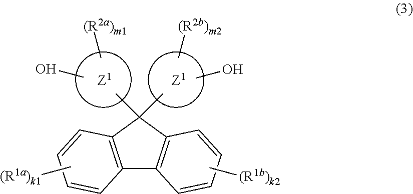
wherein in formula (3), rings Z¹, R^{1a}, R^{1b}, R^{2a}, R^{2b}, k1, k2, m1, and m2 are as defined above, 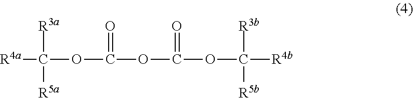
wherein in formula (4), R^{3a}, R^{3b}, R^{4a}, R^{4b}, R^{5a}, and R^{5b} are as defined above.
